(19) United States
(12) Patent Application Publication
(10) Pub. No.: US 2022/0177787 A1
(43) Pub. Date: Jun. 9, 2022

(54) NEUTRALIZATION OF HYDROGEN SULFIDE AND LIGHT MERCAPTANES IN HYDROCARBON MEDIA

(71) Applicant: Lyra Energy SRL, Worthing (BB)

(72) Inventors: Konstantin Poukalov, SR., Sarasota, FL (US); Alexander Semenyuk, Moscow (RU)

(21) Appl. No.: 17/680,675

(22) Filed: Feb. 25, 2022

Related U.S. Application Data
(63) Continuation of application No. 15/850,055, filed on Dec. 21, 2017, now abandoned.
(60) Provisional application No. 62/439,294, filed on Dec. 27, 2016.

(51) Int. Cl. C10G 45/04; C01B 21/50; C07C 215/08; C08G 75/14

(52) U.S. Cl. CPC C10G 45/04; C01B 21/50; C10G 2300/202; C08G 75/14; C10G 2300/207; C07C 215/08

(57) ABSTRACT

Disclosed is a composition and method for neutralization of hydrogen sulfide and light mercaptanes from hydrocarbon media, and improvement of the copper strip indicator in petroleum products. The composition is an aqueous solution of polysulfides comprising alkali metals and/or polysulfides of primary or secondary ethanolamines, alkali metal hydroxides, water-soluble alkylamines and optionally alkali metal nitrites.

This application is a continuation of U.S. application Ser. No. 15/850,055, filed Dec. 21, 2017, which claims the benefit of priority to U.S. provisional application Ser. No. 62/439,294, filed Dec. 27, 2016, the contents of which are incorporated herein by reference in their entirety.

### FIELD OF THE INVENTION

The invention relates to compositions and methods for neutralization of hydrogen sulfide and mercaptanes in hydrocarbon media, in particular oil, and also for improvement in the indicator using copper strips corrosion for petroleum distillates, and may be used in the oil drilling and oil refining industries. The composition for neutralization of hydrogen sulfide and light mercaptanes comprises an aqueous solution of alkali metal polysulfides and/or ethanolamine polysulfides, water-soluble aliphatic amines, an inorganic base, and optionally an alkali metal nitrite.

### BACKGROUND

Methods for removing hydrogen sulfide and mercaptanes based on use of formaldehyde or its derivatives with alcohols and/or ureic, ammonium or organic amines are known and widely used (RU 2160761, RU 2348679, RU 2118649, US 20130126429 A1, RU 2107085, RU 2246342, RU 2318864, RU 2470988 etc.). The overall flaw of the methods is ecological and sanitary problems, linked with use of highly toxic formaldehyde, identified as a carcinogen, and formation of foul-smelling reaction products of a thyolic nature (polythiomercaptals and thiomercaptals producing methylenedithol).

In recent times, methods for purification using reagents have become widely used which are based on polycondensation products of aldehydes and amine-triazines (RU 2459861, US 2008053920, US 20110220551 A1, US 20080056974 A1, U.S. Pat. Nos. 4,978,512, 7,438,877, 8,512,449 B1, etc.). The drawback of these methods is the relatively high costs of reagents. In addition, what is generally defective about all the reagents mentioned above is the presence of poorly dissolved reaction products (dithiazines, tritiane, polythiomethylenes) which form deposits difficult to remove in pipes and storage containers, and also insufficient effectiveness in regard to mercaptanes.

Methods are known using aldehydes other than formaldehyde-acroleine (EP 2367611 A1, U.S. Pat. No. 8,354,087) and glyoxal (US 20120241361 A1, US 20120329930, U.S. Pat. No. 4,680,127, RU 2499031). Acroleine is more costly than formaldehyde and extremely toxic. Use of glyoxal causes corrosion problems. In addition, such reagents are effective in regard to hydrogen sulfide, but ineffective in regard to mercaptanes.

Reagent methods based on catalytic oxidation of mercaptanes to disulfides in the presence of oxygen in air (RU 2408426, EA 018297, RU 2167187 etc.) may be effective both in regard to hydrogen sulfide and to mercaptanes. However, use of oxygen in air results in losses of light hydrocarbons removed from the raw material along with the spent air. Oxidation using aqueous solutions of hydrogen peroxide (RU 2177494, RU 2121491, RU 2146693) has its own drawbacks connected with hazards in decomposing hydrogen peroxide in the presence of bases containing nitrogen, occurring with release of oxygen in a hydrocarbon medium, and also technical difficulties of storing and using concentrated hydrogen peroxide. The aqueous-alkaline extraction methods used in industry with catalytic regeneration of alkali by oxygen from air (processes of the Merox, Mericat, DMD and DMS types) have limited applicability due to formation of water-petroleum emulsions that are difficult to separate. In addition, the difficulties in utilizing the discharges is a general drawback of such methods. Extraction processes (U.S. Pat. Nos. 2,437,348, 2,585,284, 2,309,651) are known based on use of mixtures of aqueous alkali and polar solvents (methanol, acetone, ethylene glycol and its ethers, etc.) in which regeneration (removal of mercaptanes from the extract) is carried out by distillation, frequently using steam. Such methods of extract regeneration are obsolete, and at present are not used due to unwieldiness and technological inconvenience. More modern methods which employ a mixture of aqueous alkali and ethanol (U.S. Pat. No. 1,285,043) with catalytic oxidative regeneration of the extract have as their drawback the problem of loss of ethanol with the spent air.

A method of purifying hydrocarbons is known from RU 016758 from a compound containing alcoxide or hydroxide of quaternary ammonia in the presence of metal in a high degree of oxidation, such as cobalt, iron, chromium and/or nickel. Metal in a high degree of oxidation is an oxidant, and it can be used as a catalyst in the presence of ethoxide or hydroxide of quaternary ammonia. In spite of a relative improvement in indices of depth of purification from mercaptanes due to conducting the purification in the presence with a high degree of oxidation as compared with known purification methods using only hydroxides of quaternary ammonia (U.S. Pat. Nos. 5,840,177 and 6,013,175), this method does not do away with the main drawbacks associated with use of quaternary ammonium bases—a high specific consumption of expensive reagent and neutralization of mercaptanes proceeding at an insufficient rate.

Methods are known for purification based on cholines or choline hydroxides (U.S. Pat. Nos. 4,594,147, 4,867,865), 5,183,560). The main drawback of such methods is formation of volatile sulfur-containing reaction products, and they have not found widespread acceptance in industry. Compositions based on aqueous solutions of alkalis and nitrites (RU 2230095, RU 2263705) have their own drawback in forming deposits of elemental sulfur, and they are insufficiently effective in regard to mercaptanes.

Methods are known for removing hydrogen sulfide and mercaptanes employing additional introduction of elemental sulfur into the raw material (RU 2202595, RU 2,095,393, RU 2233863, RU 2121491, RU 2167187). The common drawback of such methods is the technologically awkward operation of introducing sulfur, a difficult-to-dissolve element, into the raw material, and also contamination of the purified raw material by the added sulfur. No data are to be had on industrial application of such methods.

A method is known from RU 2252949 for petroleum purification, according to which a sulfur-containing, inorganic neutralizing reagent is used for processing, in which, in the capacity of the sulfur-containing inorganic reagent, use is made of an aqueous solution of pyrosulfite or hydrosulfite of an alkali metal, preferably sodium, or an ammonium hydrosulfite, and the process is carried out in the presence of an aqueous solution of hydroxide, orthophosphate and/or alkali metal sulfite, preferably sodium, or ammonia. The main drawback of this method is inadequate removal of mercaptanes out of the raw material, and also a relatively large consumption of the aqueous solution, which results in an undesired water contamination of the processed raw material, especially for instances with a high hydrogen sulfide content and, correspondingly, with introduction of large quantities of the aqueous phase into the oil.

Other purification methods are also known: based on maleimides (U.S. Pat. No. 4,569,766), azodicarboxilates (EP 2274400, US 20090255849), hydroquinones (US 20110315921 A1), quaternary ammonium salts (RU 2499031, U.S. Pat. No. 5,840,177, US 20080230445 A1, U.S. Pat. No. 5,840,177), aminoethers (RU 2349627). However, such reagents are rather expensive to produce, and have not been widely accepted for use in industry.

Use of solutions of alkali metal nitrites as an oxidant is known by the method of removing hydrogen sulfides from oil from RU 2230095. According to this method, purification is conducted by processing the initial raw material by an aqueous-alkaline solution of water-soluble salt of nitrous acid, preferably nitrite of alkali metal or ammonia, with a pH not less than 10 and a nitrite concentration in the solution of 3-40%. Used as the alkali agent of the aqueous-alkaline nitrite solution is a water-soluble organic amine (alkanolamine), and/or ammonia, and/or sodium hydroxide. In purification of oil containing hydrogen sulfide and mercaptanes, additionally compressed air is introduced in amounts of 0.06-0.12 nm³ to 1 mole of hydrogen sulfide and 2 moles of light methyl and ethyl mercaptanes and an aqueous or aqueous-alkaline solution of salt or metal complex of variable valence, preferably taken from computation of 0.1-1.5 g of metal ions to 1 ton of raw material. The process is conducted at a pressure of 0.2-1 MPa. The drawback of this method in particular is the need to use air and metallic compounds of variable valence for oxidation of mercaptanes: with the spent air and vapors from oil, light fractions are removed, which degrades the quality of the oil, while use of metallic complexes results in contamination of subcommercial water by heavy metal compounds. What is technically closest to the achievable result is a composition of neutralizer of hydrogen sulfide and mercaptanes for which application was made in RU 2241018. This composition contains 16-35% of alkali metal nitrite, 3-30% of a base containing nitrogen and/or alkaline reagent, and up to 100% water. In the capacity of alkali metal nitrite it preferably contains sodium nitrite, while as the base containing nitrogen and alkaline reagents it contains—alkanolamine (mono-, triethanolamine, methyldiethanolamine), and/or ammonia, and/or sodium hydroxide and potassium.

Particularly mentioned in RU 2548655 is the main drawback of the neutralizer, used in both the patents named above, which is insufficient activity in regard to mercaptanes, and also formation of elemental sulfur as a reaction product of the reagent with hydrogen sulfide. Formation of elemental sulfur results in corrosion and formation of undesired deposits on containers and pipes.

Currently as previously, there is a need for effective, accessible and inexpensive reagents for neutralizing hydrogen sulfide and mercaptanes.

### SUMMARY OF THE INVENTION

The objective of the present invention is to overcome the drawbacks mentioned above and to conduct the purification without formation of elemental sulfur and corrosive elements in the raw material. Additionally, the objective of the invention is to increase the effectiveness of the process by reducing consumption of reagent, reducing the overall quantity of the aqueous phase added to the raw material, usage of accessible reagents, and using a single stage to simultaneously remove hydrogen sulfide and mercaptanes. Another objective of the invention is to improve the copper strip indicator for petroleum distillates, which characterizes corrosion qualities of the fuel as per the standards of ASTM D130-12 or GOST 6321-92: “Fuel for engines. Test method on copper strips.”

The established goal is attained by this invention.

The invention provides chemical compositions for neutralization of hydrogen sulfide and mercaptanes in hydrocarbon media, in particular oil, and also the improvement in the indicator using copper strips corrosion for petroleum distillates, and may be used in the oil drilling and oil refining industries. The composition for neutralization of hydrogen sulfide and light mercaptanes comprises an aqueous solution of alkali metal polysulfides and/or ethanolamine polysulfides (primary or secondary) 3-15% by weight, water-soluble aliphatic amines, 2-7% by weight, and an inorganic base, 5-35% by weight. To reduce processing time and increase the reserves of reagent, the composition may also contain 5-35% by weight of an oxidant of alkali metal nitrite.

Primarily used as water-soluble aliphatic amines are alkanolamines, mainly ethanolamines, and/or diamines. Primarily used as the inorganic base are alkali metal hydroxides, preferably sodium or potassium. Preferably used as the alkali metal nitrite is sodium nitrite. The result is an increase in reagent effectiveness in neutralizing hydrogen sulfide and light mercaptanes in petroleum and petroleum products, reduction in acidity and corrosiveness of the purified raw material, including the indicator on copper strips, and also an enlarged assortment of available and inexpensive chemical reagent neutralizers for industrial purification of oils containing hydrogen sulfide and mercaptanes, and for improvement in petroleum distillate quality.

### DETAILED DESCRIPTION

In the composition for neutralization of hydrogen sulfide and light mercaptanes and hydrocarbon media, and improvement of the copper strip indicator in petroleum products containing a composition of an aqueous solution consisting of alkali metal compounds and agents containing the amino group, according to the invention, in the capacity of alkali metal compounds, the composition includes polysulfides of alkali metals and/or polysulfides of primary or secondary ethanolamines, alkali metal hydroxides, while in the capacity of agents containing the amino group, it contains water-soluble alkylamines with the following ratio of components:

polysulfides of alkali metals and/or polysulfides of primary or secondary ethanolamines of 3-15% by weight,

alkali metal hydroxides 5-35% by weight,

water soluble alkylamines 2-7% by weight,

water—the remainder.

The composition may additionally contain alkali metal nitrite in an amount of 5-35% by weight.

The composition in the capacity of alkali metal hydroxides may contain sodium or potassium hydroxides.

In its capacity as water-soluble alkylamines, the composition may contain alkanolamines and/or diamines.

In its capacity of alkali metal nitrite, the composition may contain sodium nitrite.

In its capacity of alkanolamines, the composition may contain primary, secondary or tertiary ethanolamines.

The neutralizer indicated may be applied by the reagent method (introduction into the raw material flow) jointly with any appropriate PAV known from the prior art (sulfonol, neonol, OP-10, aminoxide etc.) for improvement of the dispersion capacity in the petroleum media, or without PAV. The neutralizer may be jointly utilized with organic polar solvents known from prior art, which improve the transition of mercaptanes to the polar phase (methanol, isopropanol, glycols and their ethers, etc.) or without solvents.

The component for neutralization of hydrogen sulfide and mercaptanes is prepared by simply dissolving the components in water or mixing of their aqueous solutions under normal conditions at room temperature.

The problem posed is solved in that the neutralizer consists of an aqueous solution of the following components with content in weight %:

TABLE 1

| | |
|---|---|
| Polysulfides of alkali metals and/or ethylamines | 3-15% |
| (primary or secondary) | |
| Aliphatic amines | 2-7% |
| Inorganic base | 5-35% |
| Water | remainder |

If the raw material is processed with a high hydrogen sulfide and mercaptane content, to reduce the processing time it is appropriate to introduce alkali metal nitrite into the composition of the solution, as an oxidant, with content in weight %:

TABLE 2

| | |
|---|---|
| Polysulfides of alkali metals and/or ethylamines | 3-15% |
| (primary or secondary) | |
| Aliphatic amines | 2-7% |
| Inorganic base | 5-35% |
| Alkali metal nitrite | 5-35% |
| Water | remainder |

With this, any alternative reagent may be used for purposes of purification of raw material containing only hydrogen sulfide or only mercaptanes, and for purification of raw material that contains hydrogen sulfide and mercaptanes.

In the capacity of water-soluble aliphatic amines, preferably alkanolamines are used, preferably ethanolamines and/or diamines.

Amine polysulfides and alkali metal polysulfides used in this invention are water-soluble compounds containing a simple (non-branched) chain of sulfur atoms Sn²⁻, where n=2, 3, 4, 5 or 6. With this, the most widespread and stable compounds are deemed to be polysulfides S₂²⁻, S₄²⁻, although the polysulfides S₃²⁻, S₅²⁻, S₆²⁻ may also be present in the overall weight. Thus, sodium polysulfide is a compound with the overall formula of NaSnNa, while monoethanolamine polysulfide is HOCH₂CH₂NH₃ SₙNH₃CH₂CH₂OH. Polysulfides may be obtained by methods known in the prior art in situ, for example, by fusion of elemental sulfur and alkali, or by dissolving elemental sulfur into ethanolamine while heating, or be obtained in the form of ready-made substances for sale on the market.

A change in the share of components that goes beyond the limits indicated above, leads to a degradation of the result or to unproductive consumption of reagent. An increase in the share of inorganic base salts or alkali metal nitrite above the limits indicated above results in problems with the solubility of components, and an undesired growth in solution density.

The proposed neutralizer of hydrogen sulfide and mercaptanes under normal conditions is a uniform mobile liquid whose color is light yellow to saturated yellow in color, with a density within the limits of 1.05-1.34 g/cm³, and a hydrogen indicator of pH 11.0 and greater (depending on the content of the inorganic base, alkylamine and polysulfides).

For better comprehension, the invention may be illustrated, but not exhaustively, by the following non-limiting examples of its specific implementation.

### EXAMPLES

### Example 1

In preparing the neutralizer, dry reagents are added to the solution sequentially after all the previous additives have dissolved. The liquid reagents are added after the dry reagents have dissolved. Mix until a uniform product is obtained. All preparation is done at room temperature.

Into a vessel equipped with a mechanical mixer, 79 g of water is added, and 14 g of sodium hydroxide is loaded into it, after dissolving, 4 g of sodium polysulfide is added, after dissolving 3 g of diethanolamine is added and mixed until a uniform product is obtained. The obtained composition A1 with a content by weight % of sodium polysulfide 4, potassium hydroxide 14, diethanolamine 5, and water—remainder, is used for neutralization of hydrogen sulfide and light mercaptanes.

Similarly, other compositions are also obtained, with weight by %:
A2: sodium polysulfide—4, potassium hydroxide—14, sodium nitrate—10, diethanolamine—5, water—remainder.
A3: monoethanolamine polysulfide—8, potassium hydroxide—11, sodium nitrate—10, amylamine—4, water—remainder.
A4: diethanolamine polysulfide—10, sodium hydroxide—25, monoethanolamine—6, Water—remainder.
P (prototype, RU 2241018): sodium hydroxide—5, monoethanolamine—6, sodium nitrite—20, water—remainder.
A6: diethanolamine polysulfide—6, sodium hydroxide—14, ethylenediamine—7, water—remainder.
A7: diethanolamine polysulfide—6, sodium hydroxide—14, ethylenediamine—20, water—remainder.
A8: potassium polysulfide—5, sodium hydroxide—10, diethylamine—5, sodium nitrite—10, water—remainder.
A9: potassium polysulfide—0.5, sodium hydroxide—10, diethylamine—5, sodium nitrite—10, water—remainder.
A10: potassium polysulfide—5, sodium hydroxide—1, diethylamine—5, sodium nitrite—10, water—remainder.
A11: potassium polysulfide—5, sodium hydroxide—1, diethylamine—0.5, sodium nitrite—10, water—remainder.

### Examples 2-25

In the examples presented, a test of the composition for effectiveness in neutralizing hydrogen sulfide and light methyl and ethyl mercaptanes is conducted for the following raw-material products.
High-sulfur-content H1 oil, containing hydrogen sulfide—86 ppm, total methyl and ethyl mercaptanes—214 ppm.
High-sulfur—content H2 oil, containing hydrogen sulfide, 256 ppm, methyl and ethyl mercaptanes—not present.
GC gas condensate, hydrogen sulfide content—2 ppm, total methyl and ethyl mercaptanes—1325 ppm.
Benzene fraction of cracking BC (n.c.—205° C.), hydrogen sulfide content—120 ppm, total methyl and ethyl mercaptanes—457 ppm, test on copper strip as per GOST 6321-92—did not pass (class 3 A).

The test of the composition for effectiveness in neutralizing hydrogen sulfide and light methyl and ethyl mercaptanes in the raw material is conducted in an unheated reaction flask incorporating a mixer at room temperature. The calculated amounts of raw material and neutralizer are placed in the flask. The mass of the raw material and neutralizer is determined by weight. After the calculated time, the mixer is stopped and the sample is removed for analysis. The experimental results are presented in Table 3.

TABLE 3

| Example | Neutralizer, | Raw material | Processing time, | Processing time, | Test on copper |
|---|---|---|---|---|---|
| Number | dosage | product | result (ppm) | result (ppm) | strip |
| 1 | A1, 400 g/τ | H1 | 2 hours, | 14 hours, | |
| | | | H2S = none. | H2S = none | |
| | | | RSH = 101 | RSH = 12 | |
| 2 | A1, 320 g/τ | H2 | 2 hours, | 14 hours | |
| | | | H2S = 132. | H2S = none | |
| 3 | A1, 2000 g/τ | G | 4 hours, | 14 hours, | |
| | | | H2S = none | H2S = none | |
| | | | RSH = 550 | RSH = 21 | |
| 4 | A2, 400 g/τ | H1 | 2 hours, | 14 h [symbol] ., | |
| | | | H2S = none | H2S = none | |
| | | | RSH = 94 | RSH = 8 | |
| 5 | A2, 320 g/τ | H2 | 2 hours, | 14 hours, | |
| | | | H2S = 101 | H2S = none | |
| 6 | A2, 2000 g/τ | G | 4 hours, | 24 hours, | |
| | | | H2S = none. | H2S = none | |
| | | | RSH = 376 | RSH = 2 | |
| 7 | A3, 400 g/τ | H1 | 2 hours, | 14 h [symbol] ., | |
| | | | H2S = 12 | H2S = none | |
| | | | RSH = 87 | RSH = 21 | |
| 8 | A3, 320 g/τ | H2 | 2 hours, | 14 hours, | |
| | | | H2S = 122 | H2S = none. | |
| 9 | A3, 2000 g/τ | G | 7 hours, | 7 hours, | |
| | | | H2S = none | H2S = none | |
| | | | RSH = 453 | RSH = 28 | |
| 10 | A4, 500 g/τ | H1 | 1 hour, | 7 hours, | |
| | | | H2S = none | H2S = none | |
| | | | RSH = 91 | RSH = 2 | |
| 11 | P, 500 g/τ | H1 | 1 hour, | 14 hours, | |
| | | | H2S = 56 | H2S= none | |
| | | | RSH = 181 | RSH = 66 | |
| 12 | A5, 500 g/τ | H1 | 1 hour, | 14 hours, | |
| | | | H2S = 44 | H2S = none | |
| | | | RSH = 114 | RSH = none | |
| 13 | A4, 700 g/τ | BC | 3 hours, | 12 hours, | Passed |
| | | | H2S = none | H2S = none | (Class 1A) |
| | | | RSH = 117 | RSH = 11 | |
| 14 | P, 700 g/τ | BC | 3 hours, | 12 hours, | Did not pass |
| | | | H2S = 51 | H2S = none | (class 2B) |
| | | | RSH = 328 | RSH = 125 | |
| 15 | A5, 700 g/τ | BC | 3 hours, | 12 hours, | Passed |
| | | | H2S = none | H2S = none | (Class 1A) |
| | | | RSH = 224 | RSH = 16 | |
| 16 | A6, 700 g/τ | BC | 3 hours, | 12 hours, | Passed |
| | | | H2S = none | H2S = none | (Class 1A) |
| | | | RSH = 217 | RSH = 23 | |
| 17 | A7, 700 g/τ | BC | 3 hours, | 12 hours, | Passed |
| | | | H2S = none | H2S = none | (Class 1A) |
| | | | RSH = 208 | RSH = 21 | |
| 18 | A8, 2000 g/τ | G | 7 hours, | 16 hours, | |
| | | | H2S = none | H2S = none | |
| | | | RSH = 420 | RSH = 21 | |
| 19 | A9, 2000 g/τ | G | 7 hours, | 16 hours, | |
| | | | H2S = none | H2S = none | |
| | | | RSH = 510 | RSH = 112 | |
| 20 | A10, 2000 g/τ | G | 7 hours, | 16 hours, | |
| | | | H2S = 1 | H2S = none | |
| | | | RSH = 890 | RSH = 670 | |
| 21 | A11, 2000 g/τ | G | 7 hours, | 16 hours, | |
| | | | H2S = 1 | H2S = none | |
| | | | RSH = 608 | RSH = 420 | |
| 22 | A8, 290 g/τ | H2 | 1 hour, | 15 hours, | |
| | | | H2S = 155 | H2S = none | |
| 23 | A9, 290 g/τ | H2 | 1 hour, | 15 hours, | |
| | | | H2S = 196 | H2S = 78 | |
| 24 | A8, 720 g/τ | BC | 3 hours, | 12 hours, | Passed |
| | | | H2S = none | H2S = none | (Class 1A) |
| | | | RSH = 110 | RSH = none | |
| 25 | A9, 720 g/τ | BC | 3 hours, | 12 hours, | Did not pass |
| | | | H2S = none | H2S= | (class 3A) |
| | | | RSH = 211 | RSH = 82 | |

The examples presented demonstrate that it is possible to purify the raw material from mercaptanes only, or separately from hydrogen sulfide, or do simultaneous removal of hydrogen sulfide and mercaptanes.

Examples 10-15 show the improvement in results of processing as compared with the prototype both of the level of reduction in mercaptanes and hydrogen sulfide, and in testing of corrosion on a copper strip.

Examples 16-17 show that the increase in alkylamine content beyond the limits indicated for this invention, does not bring about a substantial improvement in the result.

Examples 1-6 show that addition of sodium nitrite into the composition results in a reduced processing time.

Examples 18-25 show that reduction in the content of a component below the limits indicated for this invention (potassium disulfide in examples 18, 19, 22, 23, 24, sodium hydroxide in example 20, and alkylamine in example 21) results in impairment in purification and the copper strip corrosion indicator.

1. A composition for neutralization of hydrogen sulfide and light mercaptans from hydrocarbon media, the composition comprising:
3-15% by weight of polysulfides of alkali metals and/or polysulfides of primary or secondary ethanolamines,
5-35% by weight of alkali metal hydroxides,
2-7% by weight of water soluble alkylamines, and
water.

2. The composition according to claim 1, further comprising 5-35% weight of an alkali metal nitrite.

3. The composition according to claim 1, wherein the alkali metal hydroxide is selected from contains sodium hydroxide and/or potassium hydroxide.

4. The composition according to claim 1, wherein the water-soluble alkylamines is selected from alkanolamines and/or diamines.

5. The composition according to claim 2, wherein the alkali metal nitrite comprises sodium nitrite.

6. The composition according to claim 4, wherein the alkanolamine comprises one or more primary, secondary or tertiary ethanolamine.
